(19) United States
(12) Patent Application Publication
(10) Pub. No.: US 2026/0184560 A1
(43) Pub. Date: Jul. 2, 2026

(54) NANOSCALE GEOMETRICAL QUANTITY CERTIFIED REFERENCE MATERIAL, AND PREPARATION METHOD AND USE THEREOF

(71) Applicant: National Institute of Metrology, China, Beijing (CN)

(72) Inventors: Yushu SHI, Beijing (CN); Jianqiao LI, Beijing (CN); Shi LI, Beijing (CN); Shu ZHANG, Beijing (CN); Fang WANG, Beijing (CN)

(21) Appl. No.: 19/431,071

(22) Filed: Dec. 23, 2025

(30) Foreign Application Priority Data
Dec. 26, 2024 (CN) 202411931136.4

(51) Int. Cl. B81C 1/00; C07C 63/333; C07D 213/90

(52) U.S. Cl. CPC B81C 1/00031; C07C 63/333; C07D 213/90; B81C 2201/0149; B81C 2201/115

(57) ABSTRACT

Provided are a nanoscale geometrical quantity certified reference material, and a preparation method and use thereof, belonging to the technical field of nano-scale measurements. The nanoscale geometrical quantity certified reference material includes a substrate and a periodic nanostructured two-dimensional supramolecular film attached to the substrate, where the periodic nanostructured two-dimensional supramolecular film is formed by self-assembly of 5,5′-(9H-fluorene-2,7-diyl)diisophthalic acid and 1,2-bis(4-pyridyl)ethane. Provided is also a preparation method of the nanoscale geometrical quantity certified reference material. The self-assembly is a bottom-up process to prepare a periodic nanostructured two-dimensional supramolecular film.

### CROSS REFERENCE TO RELATED APPLICATION

This application claims the benefit of, and priority to, Chinese Patent Application No. 202411931136.4, filed Dec. 26, 2024, which is hereby incorporated by reference herein in its entirety.

### TECHNICAL FIELD

The present disclosure relates to the technical field of nano-scale measurements, in particular a nanoscale geometrical quantity certified reference material, and a preparation method and use thereof.

### BACKGROUND

As nodes in the integrated circuit manufacturing process continuously shrink, chip performance is increasingly affected by geometric quantities of nano-dimensions, which poses new requirements on high precision and stability in nanometrology. Conventional nano-measurement technologies primarily rely on laser interferometry, which measures changes in interference fringes to infer minute displacements or length changes of an object to be measured. Nonlinear errors may occur in laser interferometry due to various factors (such as errors in optical components and signal distortion), adversely affecting the accuracy and stability of measurement. Furthermore, laser interferometry is sensitive to environmental conditions (such as temperature, humidity, and vibration), and thus measurements need to be performed under relatively stable environmental conditions, and corresponding measures are needed to be taken to reduce the impact of environmental factors, so the maintenance costs are high. Therefore, laser interferometry does not have advantages in terms of measurement accuracy, stability, and measurement cost of nano-measurement.

In 2018, the Bureau International des Poids et Mesures stipulated silicon lattice constant as the method for reproducing the definition of the International System of Units (SI) meter, and nanoscale geometrical quantity certified reference materials can be traced not only to laser wavelength but also to atomic-scale silicon lattice constants. This method improves the accuracy in determined values by nanoscale geometrical quantity certified reference materials, bringing about huge advantages in the traceability and calibration of nanometrology. Nowadays, the methods for preparing a nanoscale geometrical quantity certified reference material primarily include photolithography, electron beam lithography, deposition and etching technologies, and so on. The precision of nanoscale geometrical quantity certified reference materials prepared using the above methods needs to be further improved.

### SUMMARY OF THE INVENTION

In view of this, an object of the present disclosure is to provide a nanoscale geometrical quantity certified reference material, and a preparation method and use thereof. The nanoscale geometrical quantity certified reference material provided by the present disclosure has excellent precision.

To achieve the foregoing object, the present disclosure provides the following technical solutions.

The present disclosure provides a nanoscale geometrical quantity certified reference material including a substrate and a periodic nanostructured two-dimensional supramolecular film attached to the substrate, wherein the periodic nanostructured two-dimensional supramolecular film is formed by self-assembly of 5,5′-(9H-fluorene-2,7-diyl)diisophthalic acid and 1,2-bis(4-pyridyl)ethane.

In some embodiments, the substrate is made of a material including one selected from the group consisting of highly oriented pyrolytic graphite, gold, and copper.

The present disclosure further provides a preparation method of the nanoscale geometrical quantity certified reference material as described in the above technical solutions, including:
providing a substrate; and
self-assembling the 5,5′-(9H-fluorene-2,7-diyl)diisophthalic acid and the 1,2-bis(4-pyridyl)ethane on the substrate to obtain the nanoscale geometrical quantity certified reference material.

In some embodiments, the self-assembling the 5,5′-(9H-fluorene-2,7-diyl)diisophthalic acid and the 1,2-bis(4-pyridyl)ethane on the substrate includes: sequentially adding a 5,5′-(9H-fluorene-2,7-diyl)diisophthalic acid solution and a 1,2-bis(4-pyridyl)ethane solution dropwise onto the substrate, and conducting self-assembly.

In some embodiments, the self-assembling the 5,5′-(9H-fluorene-2,7-diyl)diisophthalic acid and the 1,2-bis(4-pyridyl)ethane on the substrate includes: sequentially adding a 1,2-bis(4-pyridyl)ethane solution and a 5,5′-(9H-fluorene-2,7-diyl)diisophthalic acid solution dropwise onto the substrate, and conducting self-assembly.

In some embodiments, the self-assembling the 5,5′-(9H-fluorene-2,7-diyl)diisophthalic acid and the 1,2-bis(4-pyridyl)ethane on the substrate includes: dropwise adding a mixed solution of the 5,5′-(9H-fluorene-2,7-diyl)diisophthalic acid and the 1,2-bis(4-pyridyl)ethane onto the substrate, and conducting self-assembly.

In some embodiments, the mixed system formed from the 5,5′-(9H-fluorene-2,7-diyl)diisophthalic acid solution and the 1,2-bis(4-pyridyl)ethane solution, a molar ratio of the 5,5′-(9H-fluorene-2,7-diyl)diisophthalic acid to the 1,2-bis(4-pyridyl)ethane is in a range of 1:1 to 1.5:1, a 5,5′-(9H-fluorene-2,7-diyl)diisophthalic acid concentration is in a range of 0.0005-0.002 mol/mL, and a pH is in a range of 6-7.5.

In some embodiments, in the mixed solution of the 5,5′-(9H-fluorene-2,7-diyl)diisophthalic acid and the 1,2-bis(4-pyridyl)ethane, a molar ratio of the 5,5′-(9H-fluorene-2,7-diyl)diisophthalic acid to the 1,2-bis(4-pyridyl)ethane is in a range of 1-1.5:1, a 5,5′-(9H-fluorene-2,7-diyl)diisophthalic acid concentration is in a range of 0.0005-0.002 mol/mL, and a pH is in a range of 6-7.5.

In some embodiments, the preparation method further includes: mechanically exfoliating the substrate before use.

The present disclosure further provides use of the nanoscale geometrical quantity certified reference material as described in the above technical solutions or the nanoscale geometrical quantity certified reference material obtained by the method as described in the above technical solutions in nanometer measurement.

The present disclosure provides a nanoscale geometrical quantity certified reference material.

In the present disclosure, a periodic nanostructured two-dimensional supramolecular film is formed on a substrate by self-assembly of 5,5′-(9H-fluorene-2,7-diyl)diisophthalic acid and 1,2-bis(4-pyridyl)ethane. The self-assembled periodic nanostructured two-dimensional supramolecular film has excellent precision.

The present disclosure also provides a preparation method of the nanoscale geometrical quantity certified reference material as described in the above technical solutions. The self-assembly is a bottom-up process to prepare a periodic nanostructured two-dimensional supramolecular film. This bottom-up process allows for more precise control of the morphology, size, and properties of the nanostructure, so that the periodic nanostructured two-dimensional supramolecular film has high precision and excellent performance. By controlling the self-assembly conditions, the length, linewidth, and duty cycle of the geometric nanostructure can be modulated. Furthermore, the preparation method of the present disclosure produces a cross-scale geometric nanostructure. Additionally, the preparation method of the present disclosure has the advantages of simple operations, low cost and easy scale-up, and is suitable for large-scale production applications.

### BRIEF DESCRIPTION OF THE DRAWINGS

FIG. 1 shows a flow chart of the first self-assembly according to an embodiment of the present disclosure. In FIG. 1, S1: preparing a 5,5′-(9H-fluorene-2,7-diyl)diisophthalic acid solution and a 1,2-bis(4-pyridyl)ethane solution; S1.1: mechanically exfoliating a substrate; S2: dropwise adding the 5,5′-(9H-fluorene-2,7-diyl)diisophthalic acid solution onto a surface of the substrate; and S3: dropwise adding the 1,2-bis(4-pyridyl)ethane solution.

FIG. 2 is a scanning tunneling microscope (STM) image of nanogrids of the periodic nanostructured two-dimensional supramolecular film obtained in Example 1.

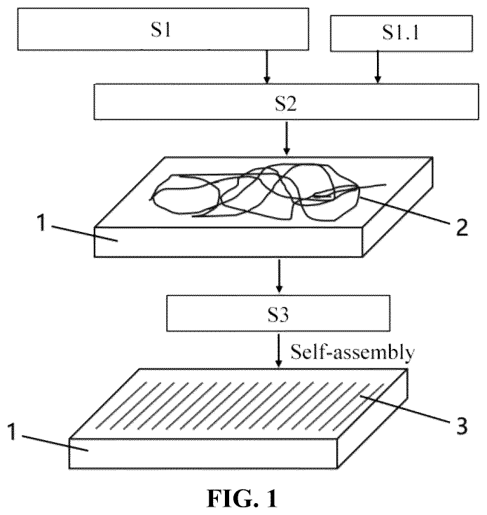

In FIGS. 1, 1 represents a substrate, 2 represents a two-dimensional supramolecular film having a disordered structure, and 3 represents a periodic nanostructured two-dimensional supramolecular film.

### DETAILED DESCRIPTION OF THE EMBODIMENTS

The present disclosure provides a nanoscale geometrical quantity certified reference material including a substrate and a periodic nanostructured two-dimensional supramolecular film attached to the substrate, wherein the periodic nanostructured two-dimensional supramolecular film is formed by self-assembly of 5,5′-(9H-fluorene-2,7-diyl)diisophthalic acid and 1,2-bis(4-pyridyl)ethane.

Unless otherwise specified, raw materials used in the present disclosure are preferably commercially available products.

The nanoscale geometrical quantity certified reference material provided in the present disclosure includes a substrate; in some embodiments, the substrate is made of a material including highly oriented pyrolytic graphite, gold or copper, preferably highly oriented pyrolytic graphite.

The nanoscale geometrical quantity certified reference material provided by the present disclosure includes a periodic nanostructured two-dimensional supramolecular film attached to the substrate, wherein the periodic nanostructured two-dimensional supramolecular film is formed by self-assembly of 5,5′-(9H-fluorene-2,7-diyl)diisophthalic acid and 1,2-bis(4-pyridyl)ethane. In some embodiments of the present disclosure, the periodic nanostructured two-dimensional supramolecular film has a thickness of 0.3-1 nm. In some embodiments of the present disclosure, the molar ratio of the 5,5′-(9H-fluorene-2,7-diyl)diisophthalic acid to the 1,2-bis(4-pyridyl)ethane in the periodic nanostructured two-dimensional supramolecular film is in a range of 1-1.5:1, specifically preferably 1:1, 1.1:1, 1.2:1, 1.3:1, 1.4:1, or 1.5:1.

In some embodiments of the present disclosure, the periodic nanostructured two-dimensional supramolecular film is attached to any one surface of the substrate. In the present disclosure, the periodic nanostructured two-dimensional supramolecular film is attached to any surface of the substrate, so that any position of the nanoscale geometrical quantity certified reference material can be used without requiring positioning.

In some embodiments of the present disclosure, a periodic nanostructure of the periodic nanostructured two-dimensional supramolecular film includes a nanometer linewidth structure or a nanogrid structure.

In some embodiments of the present disclosure, the periodic nanostructured two-dimensional supramolecular film has a geometric nanostructure across a wide range of scales; in some embodiments, the geometric nanostructure includes step height, length, pitch, and linewidth.

In the present disclosure, a periodic nanostructured two-dimensional supramolecular film is formed on a substrate by self-assembly of 5,5′-(9H-fluorene-2,7-diyl)diisophthalic acid and 1,2-bis(4-pyridyl)ethane. The self-assembled periodic nanostructured two-dimensional supramolecular film has excellent precision.

The present disclosure further provides a preparation method of the nanoscale geometrical quantity certified reference material as described in the above technical solutions, including:
providing a substrate; and
self-assembling the 5,5′-(9H-fluorene-2,7-diyl)diisophthalic acid and the 1,2-bis(4-pyridyl)ethane on the substrate to obtain the nanoscale geometrical quantity certified reference material.

The present disclosure provides a substrate. In some embodiments of the present disclosure, the substrate is made of a material consistent with that of the aforementioned technical solutions and will not be repeated here. In some embodiments of the present disclosure, before using the substrate, the preparation method further includes mechanically exfoliating the substrate. In the present disclosure, there is no specifical limitations on the parameters of the mechanical exfoliation.

In the present disclosure, after a substrate is provided, 5,5′-(9H-fluorene-2,7-diyl)diisophthalic acid and 1,2-bis(4-pyridyl)ethane are self-assembled on the substrate to obtain the nanoscale geometrical quantity certified reference material.

In some embodiments of the present disclosure, the self-assembling the 5,5′-(9H-fluorene-2,7-diyl)diisophthalic acid and the 1,2-bis(4-pyridyl)ethane on the substrate includes: sequentially adding a 5,5′-(9H-fluorene-2,7-diyl)diisophthalic acid solution and a 1,2-bis(4-pyridyl)ethane solution dropwise onto the substrate, and conducting the self-assembly (referred to as the first self-assembly). In some embodiments of the present disclosure, a solvent for the 5,5′-(9H-fluorene-2,7-diyl)diisophthalic acid solution is a polar solvent; the polar solvent preferably includes one or more of heptanoic acid, octylbenzene, and furan, further preferably heptanoic acid. In some embodiments of the present disclosure, the 5,5′-(9H-fluorene-2,7-diyl)diisophthalic acid solution has a concentration of 0.001-0.002 mol/mL, specifically preferably 0.001 mol/mL, 0.0015 mol/mL, or 0.002 mol/mL. In some embodiments of the present disclosure, the 5,5′-(9H-fluorene-2,7-diyl)diisophthalic acid solution is dropwise added in an amount of 1-2 μL/cm², specifically preferably 1 μL/cm². In some embodiments of the present disclosure, after the dropwise addition of the 5,5′-(9H-fluorene-2,7-diyl)diisophthalic acid solution is completed, the 1,2-bis(4-pyridyl)ethane solution is directly added dropwise.

In some embodiments of the present disclosure, the solvent for the 1,2-bis(4-pyridyl)ethane solution is the same as the solvent for the 5,5′-(9H-fluorene-2,7-diyl)diisophthalic acid solution, and will not be repeated here. In some embodiments of the present disclosure, the 1,2-bis(4-pyridyl)ethane solution has concentration of 0.001-0.002 mol/mL, specifically preferably 0.001 mol/mL, 0.0015 mol/mL, or 0.002 mol/mL. In some embodiments of the present disclosure, the 1,2-bis(4-pyridyl)ethane solution is dropwise added in an amount of 1-2 μL/cm², specifically preferably 1 μL/cm².

In some embodiments of the present disclosure, in the mixed system formed from the 5,5′-(9H-fluorene-2,7-diyl)diisophthalic acid solution and the 1,2-bis(4-pyridyl)ethane solution, a molar ratio of the 5,5′-(9H-fluorene-2,7-diyl)diisophthalic acid to the 1,2-bis(4-pyridyl)ethane is in a range of 1-1.5:1, specifically preferably 1:1, 1.1:1, 1.2:1, 1.3:1, 1.4:1 or 1.5:1; a 5,5′-(9H-fluorene-2,7-diyl)diisophthalic acid concentration is preferably in a range of 0.0005-0.002 mol/mL, specifically preferably 0.0005 mol/mL, 0.00075 mol/mL or 0.001 mol/mL; and a pH is preferably in a range of 6.0-7.5.

In some embodiments of the present disclosure, the first self-assembly is performed at room temperature, the first self-assembly is performed preferably for 8 to 15 minutes, specifically preferably for 10 minutes. In some embodiments, after the first self-assembly, the preparation method of the present disclosure further includes evaporating a solvent to dryness; evaporating the solvent to dryness is preferably at a temperature of 50-70° C., further preferably at 60° C. In the present disclosure, there is no any specific limitation on the time for evaporating the solvent to dryness, as long as the solvent can be completely removed.

In the present disclosure, the flow chart for the first self-assembly is shown in FIG. 1 and specifically includes: first preparing a 5,5′-(9H-fluorene-2,7-diyl)diisophthalic acid solution and a 1,2-bis(4-pyridyl)ethane solution; mechanically exfoliating a substrate 1, adding the 5,5′-(9H-fluorene-2,7-diyl)diisophthalic acid solution dropwise to a surface of the substrate 1 to form a two-dimensional supramolecular film 2 having a disordered structure; and then adding the 1,2-bis(4-pyridyl)ethane solution dropwise, and conducting self-assembly, to afford a periodic nanostructured two-dimensional supramolecular film 3.

In some embodiments of the present disclosure, the self-assembling 5,5′-(9H-fluorene-2,7-diyl)diisophthalic acid and 1,2-bis(4-pyridyl)ethane on the substrate includes: sequentially adding a 1,2-bis(4-pyridyl)ethane solution and a 5,5′-(9H-fluorene-2,7-diyl)diisophthalic acid solution dropwise onto the substrate, and conducting self-assembly (referred to as a second self-assembly).

In some embodiments of the present disclosure, the solvents, concentrations and addition amounts of the 1,2-bis(4-pyridyl)ethane solution and 5,5′-(9H-fluorene-2,7-diyl)diisophthalic acid solution are consistent with those in the above technical solutions, and will not be repeated here. In some embodiments of the present disclosure, in the mixed system formed from the 5,5′-(9H-fluorene-2,7-diyl)diisophthalic acid solution and the 1,2-bis(4-pyridyl)ethane solution, a molar ratio of the 5,5′-(9H-fluorene-2,7-diyl)diisophthalic acid to the 1,2-bis(4-pyridyl)ethane, a 5,5′-(9H-fluorene-2,7-diyl)diisophthalic acid concentration, and a pH are consistent with the above technical solutions, and will not be repeated here.

In some embodiments of the present disclosure, the temperature, time and post-treatment of the second self-assembly are consistent with the above technical solutions, and will not be repeated here.

In some embodiments of the present disclosure, self-assembling 5,5′-(9H-fluorene-2,7-diyl)diisophthalic acid and 1,2-bis(4-pyridyl)ethane on the substrate includes: dropwise adding a mixed solution of 5,5′-(9H-fluorene-2,7-diyl)diisophthalic acid and 1,2-bis(4-pyridyl)ethane onto the substrate, and conducting self-assembly (referred to as a third self-assembly). In some embodiments of the present disclosure, in the mixed solution of 5,5′-(9H-fluorene-2,7-diyl)diisophthalic acid and 1,2-bis(4-pyridyl)ethane, a molar ratio of the 5,5′-(9H-fluorene-2,7-diyl)diisophthalic acid to the 1,2-bis(4-pyridyl)ethane is in a range of 1-1.5:1, specifically preferably 1:1, 1.1:1, 1.2:1, 1.3:1, 1.4:1 or 1.5:1; a 5,5′-(9H-fluorene-2,7-diyl)diisophthalic acid concentration is in a range of 0.0005-0.002 mol/mL, specifically preferably 0.0005 mol/mL, 0.00075 mol/mL or 0.001 mol/mL; and a pH is in a range of 6.0-7.5.

In some embodiments of the present disclosure, the temperature, time and post-treatment of the third self-assembly are consistent with the above technical solutions, and will not be repeated here.

In the present disclosure, the self-assembly is a bottom-up process to prepare a periodic nanostructured two-dimensional supramolecular film. This bottom-up process allows for more precise control of the morphology, size, and properties of the nanostructure, so that the periodic nanostructured two-dimensional supramolecular film has high precision and excellent performance. By controlling the self-assembly conditions, the length, linewidth, and duty cycle of the geometric nanostructure can be modulated. Furthermore, the preparation method of the present disclosure produces a cross-scale geometric nanostructure. Additionally, the preparation method of the present disclosure has the advantages of simple operations, low cost and easy scale-up, and is suitable for large-scale production applications.

The present disclosure further provides use of the nanoscale geometrical quantity certified reference material as described in the above technical solutions or the nanoscale geometrical quantity certified reference material obtained by the method as described in the above technical solutions in nanometer measurement.

In some embodiments of the present disclosure, an application device of the nanoscale geometrical quantity certified reference material include a scanning tunneling microscope and an atomic force microscope; the nanoscale geometrical quantity certified reference material can be used for calibration and traceability of scanning tunneling microscopes and atomic force microscopes.

A nanoscale geometrical quantity certified reference material, a preparation method and use thereof provided by the present disclosure will be explained in detail with reference to the following example. However, this example should not be construed as limiting the scope of the present disclosure.

### Example 1

5,5′-(9H-fluorene-2,7-diyl)diisophthalic acid was dissolved in heptanoic acid to afford a 5,5′-(9H-fluorene-2,7-diyl)diisophthalic acid solution having a concentration of 0.001 mol/mL.

1,2-bis(4-pyridyl)ethane was dissolved in heptanoic acid to obtain a 1,2-bis(4-pyridyl)ethane solution having a concentration of 0.001 mol/mL.

A highly oriented pyrolytic graphite substrate was mechanically exfoliated to prepare a clean substrate surface.

The 5,5′-(9H-fluorene-2,7-diyl)diisophthalic acid solution was dropwise added onto the clean substrate surface in an amount of 1 μL/cm² until the 5,5′-(9H-fluorene-2,7-diyl)diisophthalic acid solution covered the substrate surface, forming a large-scale two-dimensional supramolecular film having a disordered structure.

The 1,2-bis(4-pyridyl)ethane solution was added dropwise in an amount of 1 μL/cm² onto the surface of the large-scale two-dimensional supramolecular film having a disordered structure, and self-assembly was conducted for 10 minutes. After the addition of the 1,2-bis(4-pyridyl)ethane solution, in the resulting mixed system, the 1,2-bis(4-pyridyl)ethane concentration was 0.0005 mol/mL and the 5,5′-(9H-fluorene-2,7-diyl)diisophthalic acid concentration was 0.0005 mol/mL. The pH of the resulting mixed system was 6.5, and the environment temperature was 25° C. The solvent was removed to afford a nanoscale geometrical quantity certified reference material.

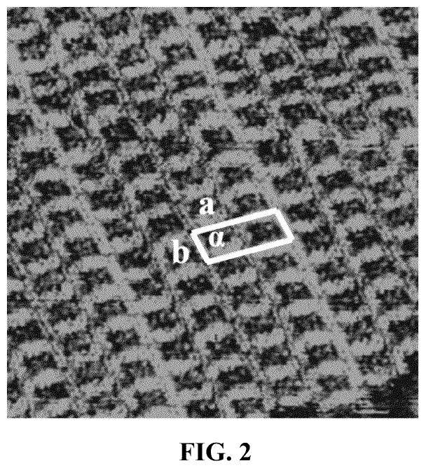

FIG. 2 shows a scanning tunneling microscope (STM) image of nanogrids of the periodic nanostructured two-dimensional supramolecular film in the resulting nanoscale geometrical quantity certified reference material. As shown in FIG. 2, the linewidth a of the grid structure in the direction A is 4.55 nm, and the linewidth b in the B direction is 1.90 nm, with uniform line widths, a neat structure, and excellent periodicity.

The preparation method of the present disclosure enables the preparation of a one-or two-dimensional standard reference grid with high precision, stable performance, and cross-scale on the substrate surface. By controlling the self-assembly, the periodicity and duty cycle of the geometric structure can be modulated, enabling precise control of one-or two-dimensional nanogrids. This complements existing one-or two-dimensional standard reference grids and promotes the rapid development of related disciplines and industries. The nanoscale geometrical quantity certified reference material prepared in the present disclosure is applicable to the calibration and traceability of scanning tunneling microscopes and atomic force microscopes.

The above example is merely a preferred embodiment of the present disclosure. It should be noted that a person of ordinary skill in the art may make some improvements and modifications without departing from the principle of the present disclosure, and these improvements and modifications should also be regarded as falling within the scope of the present disclosure.

What is claimed is:

1. A nanoscale geometrical quantity certified reference material, comprising a substrate and a periodic nanostructured two-dimensional supramolecular film attached to the substrate,
wherein the periodic nanostructured two-dimensional supramolecular film is formed by self-assembly of 5,5′-(9H-fluorene-2,7-diyl)diisophthalic acid and 1,2-bis(4-pyridyl)ethane.

2. The nanoscale geometrical quantity certified reference material as claimed in claim 1, wherein the substrate is made of a material including one selected from the group consisting of highly oriented pyrolytic graphite, gold, and copper.

3. A preparation method of the nanoscale geometrical quantity certified reference material as claimed in claim 1, comprising:
providing the substrate; and
self-assembling the 5,5′-(9H-fluorene-2,7-diyl)diisophthalic acid and the 1,2-bis(4-pyridyl)ethane on the substrate to obtain the nanoscale geometrical quantity certified reference material.

4. The preparation method as claimed in claim 3, wherein the substrate is made of a material including one selected from the group consisting of highly oriented pyrolytic graphite, gold, and copper.

5. The preparation method as claimed in claim 3, wherein the self-assembling the 5,5′-(9H-fluorene-2,7-diyl)diisophthalic acid and the 1,2-bis(4-pyridyl)ethane on the substrate comprises: sequentially adding a 5,5′-(9H-fluorene-2,7-diyl)diisophthalic acid solution and a 1,2-bis(4-pyridyl)ethane solution dropwise onto the substrate, and conducting the self-assembly.

6. The preparation method as claimed in claim 3, wherein the self-assembling the 5,5′-(9H-fluorene-2,7-diyl)diisophthalic acid and the 1,2-bis(4-pyridyl)ethane on the substrate comprises: sequentially adding a 1,2-bis(4-pyridyl)ethane solution and a 5,5′-(9H-fluorene-2,7-diyl)diisophthalic acid solution dropwise onto the substrate, and conducting the self-assembly.

7. The preparation method as claimed in claim 3, wherein the self-assembling the 5,5′-(9H-fluorene-2,7-diyl)diisophthalic acid and the 1,2-bis(4-pyridyl)ethane on the substrate comprises: dropwise adding a mixed solution of the 5,5′-(9H-fluorene-2,7-diyl)diisophthalic acid and the 1,2-bis(4-pyridyl)ethane onto the substrate, and conducting the self-assembly.

8. The preparation method as claimed in claim 5, wherein in a mixed system formed from the 5,5′-(9H-fluorene-2,7-diyl)diisophthalic acid solution and the 1,2-bis(4-pyridyl)ethane solution, a molar ratio of the 5,5′-(9H-fluorene-2,7-diyl)diisophthalic acid to the 1,2-bis(4-pyridyl)ethane is in a range of 1:1 to 1.5:1, a 5,5′-(9H-fluorene-2,7-diyl)diisophthalic acid concentration is in a range of 0.0005-0.002 mol/mL, and a pH is in a range of 6-7.5.

9. The preparation method as claimed in claim 6, wherein in a mixed system formed from the 5,5′-(9H-fluorene-2,7-diyl)diisophthalic acid solution and the 1,2-bis(4-pyridyl)ethane solution, a molar ratio of the 5,5′-(9H-fluorene-2,7-diyl)diisophthalic acid to the 1,2-bis(4-pyridyl)ethane is in a range of 1:1 to 1.5:1, a 5,5′-(9H-fluorene-2,7-diyl)diisophthalic acid concentration is in a range of 0.0005-0.002 mol/mL, and a pH is in a range of 6-7.5.

10. The preparation method as claimed in claim 7, wherein in the mixed solution of the 5,5′-(9H-fluorene-2,7-diyl)diisophthalic acid and the 1,2-bis(4-pyridyl)ethane, a molar ratio of the 5,5′-(9H-fluorene-2,7-diyl)diisophthalic acid to the 1,2-bis(4-pyridyl)ethane is in a range of 1:1 to 1.5:1, a 5,5′-(9H-fluorene-2,7-diyl)diisophthalic acid concentration is in a range of 0.0005-0.002 mol/mL, and a pH is in a range of 6-7.5.

11. The preparation method as claimed in claim 3, further comprising: mechanically exfoliating the substrate before use.

12. A use method of the nanoscale geometrical quantity certified reference material as claimed in claim 1 in nano-scale measurements.
